(19) United States
(12) Patent Application Publication
(10) Pub. No.: US 2006/0069383 A1
(43) Pub. Date: Mar. 30, 2006

(54) Guiding member for surgical instruments, surgical instruments, coupling and uses thereof

(76) Inventors: Georges Bogaerts, Brussels (BE); Andre Scattolin Faure, Besancon (FR)

Correspondence Address: MERCHANT & GOULD PC, P.O. BOX 2903, MINNEAPOLIS, MN, 55402-0903, US

(21) Appl. No.: 10/517,573

(22) PCT Filed: Jun. 26, 2003
(86) PCT No.: PCT/BE03/00113

§ 371 Date: Aug. 11, 2005

Related U.S. Application Data
(60) Provisional application No. 60/392,736, filed on Jun. 28, 2002.
(60) Provisional application No. 60/392,737, filed on Jun. 28, 2002.
(60) Provisional application No. 60/407,459, filed on Aug. 30, 2002.

(51) Int. Cl. A61B 17/00; A61F 11/00; A61B 19/00

(52) U.S. Cl. 606/1; 606/108; 128/898

(57) ABSTRACT

The present invention is related to a guiding member for guiding surgical instruments to a target volume inside a patient. The present invention also concerns surgical instruments specifically adapted for cardiac or hepatic surgery as well as a surgical assembly coupling said guiding member and said surgical instruments.

### FIELD OF THE INVENTION

As a first object of the present invention is related to a guiding member for surgical instruments which may be coupled to particular surgical instruments adapted for specific surgery applications.

In particular, said guiding member can be used for guiding the penetration of surgical instruments inside anatomic organs such as a heart or a liver, namely micro-robotic instruments according to the invention.

A second object of the invention concerns micro-robotic surgical instruments specifically adapted for cardiac surgery, and more specifically for treating atrial fibrillation and methods using them.

A third object of the present invention concerns micro-robotic surgical instruments specifically adapted for hepatic surgery, and namely the ablation of hepatic tumors and methods using them.

A fourth object of the invention concerns surgical assemblies comprising the guiding member according to the invention and the instruments according to the invention.

Other objects of the invention concern surgical assemblies comprising either the guiding member or the instruments according to the invention.

### STATE OF THE ART

Since the 1980s and the first totally laparoscopic cholecystectomy (gall bladder ablation) done by Mouret in 1987, minimally invasive techniques represent an alternative to classical surgery which combine the effects of being safe and reproducible but also of being less invasive and less traumatic for the patient than classical surgery. Said techniques also require less important post operative care than classical surgery.

In these minimally invasive techniques, a small incision is done and surgical instruments are placed at the tip of a long stem for their introduction in the patient cavity. The surgical field is shown to the surgeon by a camera also introduced in the patient cavity by a small incision so that the surgeon may visualize the whole surgery procedure on a screen linked to the camera.

In the endovascular approach, it has been proposed to use catheters mounted on flexible wires and introduced in a blood vessel to reach anatomical organs located in the circulatory system. Said catheters may be provided with cutting means such as radio-frequency stimulable electrodes so that they may induce targeted lesions at said anatomical organ or even a targeted ablation of a tissue volume at said anatomical organ.

An example of application of such catheters is atrial defibrillation (AF). Said catheters preliminary introduced in a blood vessel are directed to the heart, where located lesions in the inner atrial wall are produced in order to stop the chaotic electric pulses existing in atrial fibrillation. However, such catheters present some drawbacks. Since they are placed at the tip of a flexible wire, there is a lack for a rigid support which would allow an effective contact between the electrode and the atrial wall. Moreover, the difficulty of repositioning the catheter tip is responsible for the variable failure rate. Another drawback comes from the fact that the major part of catheter procedures are done under long X-ray exposure (between 3-6 hours). In addition, the use of intra-cardiac catheters is associated with an increased stroke risk.

Document U.S. Pat. No. 5,823,956 discloses devices adapted to work on a beating heart. In one embodiment, a tubular access device having an inner lumen is provided for positioning through a penetration in a muscular wall of the heart, said access device comprising means for sealing within the penetration, such as a balloon or flange, to inhibit leakage of blood outside the anatomic organ. An obturator is also removably positionable in the inner lumen of the access device, said obturator having cutting means, such as radiofrequency electrodes, at one of its ends for penetrating the muscular wall of the heart. Elongated instruments may be introduced through the tubular access device into an interior chamber of the heart for performing surgical procedures.

Nevertheless, if said device offers the advantage of suppressing the risks associated to the working on heart under cardioplegic arrest, its use is however still problematic. Firstly, said device does not provide sufficient stabilisation of the heart, because the access device is manipulated by the surgeon. Secondly, the use of said device still causes a non-negligible trauma for the patient as the access device penetrates into the cardiac chamber. Thirdly, for the embodiment using a balloon or flange as sealing means, the ability of the surgical instrument passing through the access device to adapt the volume of the internal wall of the cardiac chamber is limited. In other words, the positioning of the surgical instrument inside the cardiac chamber with said device is still problematic.

Another application of such catheters concerns tumour ablation, and in particular hepatic tumour ablation by radiofrequencies. The procedure offers the advantage to be very short as it lasts 10-15 minutes and the patient goes back home on the same day. Moreover, the majority of patients do not experience side effects and resume normal activity the following day. The results with this technique on small tumours are rather good. Nevertheless, this technique has still to face to the major problems of reaching the tumour and finding an adequate equilibrium between total eradication of the tumour and preservation of surrounding safe tissues. Deployable electrodes in a certain configuration have been proposed to ablate voluminous tumours but the obtained results were still unsatisfying, as the destruction of functional hepatic areas is quite important.

More generally, the use of catheters in cardiac or hepatic surgery have to face inherent problems related to minimally invasive techniques already proposed. A first problem is the difficulty to perform complex surgeries when invasiveness decreases. Another problem is the lack of 3D spatial view, since visualization is done through a camera.

In other words, in hepatic surgery as well as in cardiac surgery, there is still a need for a surgical assembly which would allow a specific treatment of a targeted tissue volume by creating lesions or even ablation, while preserving surrounding safe tissues.

### AIMS OF THE INVENTION

The present invention aims to provide an auxiliary device able to be used in combination with a surgical instrument able to penetrate inside an anatomic organ such as a heart or a liver, which does not present the drawbacks of the devices disclosed in the prior art.

In particular, the present invention aims to provide an auxiliary device consisting of a guiding member for a surgical instrument which ensures good stabilisation of the anatomic organ to be treated so as to allow the use of robotic surgical instruments.

The present invention also aims to provide a guiding member configured so as to render any position inside the anatomic organ accessible for the surgical instrument. In other words, the present invention aims to provide a guiding member which does not restrain the positioning of said surgical instrument inside the anatomic organ.

Another aim of the present invention is to provide a guiding member wherein the risk of blood leakage inside the organism is avoided.

In addition, the present invention aims to provide a guiding member the use of which minimises the trauma for the patient.

The present invention also aims to provide a surgical instrument which could be used in combination with the guiding member of the invention so as to form a new surgical assembly and which could be inserted inside an anatomic organ such as a heart or a liver.

In particular, the present invention also aims to provide a surgical instrument and method for creating lesions in a heart chamber for the treatment of atrial fibrillation, which do not present the drawbacks of the surgical instruments and methods of the state of the art.

Particularly, the present invention aims to provide an instrument and a method for treating atrial fibrillation in a beating heart.

Another aim of the present invention is to provide an instrument and method for creating lesions on a beating heart, with a millimetre precision.

Another aim of the present invention is to provide an instrument and method offering easier accessibility to the heart chambers, while being traumatic as less as possible for the patient.

The present invention also aims to provide a surgical instrument and a method for destroying specific target regions inside a liver for the treatment of hepatic cancer tumours, which do not present the drawbacks of the instruments and methods of the state of the art.

In particular, the present invention aims to provide an instrument and a method for destroying by coagulation, specific target regions inside a liver, said specific target regions preferably corresponding to hepatic tumours.

Another aim of the present invention is to provide an instrument and method for coagulating target regions in a functional liver, with a millimetre precision.

Another aim of the present invention is to provide an instrument and method offering easier accessibility to the hepatic parenchyma, while being traumatic as less as possible for the patient.

### SUMMARY OF THE INVENTION

A first object of the invention is related to a hollow guiding member for guiding a surgical instrument to a target presenting an outer surface, said target being preferably an anatomic organ such as a beating heart or a liver, said guiding member having a proximal portion and a distal portion and comprising:
at its proximal portion, an elongated rigid body having a first inner lumen;
at its distal portion, flexible sealing means mounted on said body, for sealing said guiding member on the outer surface of the target, said sealing means having a second inner lumen which communicates with the first inner lumen of the body;
   the conformation of the guiding member as a whole being such that a surgical instrument may pass through it.

Preferably, the body of said guiding member comprises a distal end and a proximal end, the distal end being connected to the sealing means and the proximal end being connected via fixation means to stabilisation means, said stabilisation means comprising immobile support means.

Preferably, said fixation means correspond to a trocar.

Advantageously, said stabilisation means comprise at least one support arm attachable to a surgical table.

Moreover, preferably, the sealing means correspond to a sucker, preferably of conical shape, having a top and a base, the top being narrower than the base and being connected to the distal end of said body, said sealing means further comprising connection means for connecting said sealing means to an external negative pressure generator.

In addition, the guiding member according to the invention may comprise a valve, preferably an homeostatic valve, disposed therein.

The present invention is also related to a method for performing a surgical intervention on a targeted anatomic organ, such as beating heart or a liver, using a surgical instrument, preferably a robotic surgical instrument, coupled to the guiding member according to any one of the preceding claims, said method comprising the following steps:
coupling the guiding member to fixation means such as a trocar;
connecting the guiding member to immobile support means via said fixation means;
connecting the sucker of the guiding member to an external negative pressure generator;
creating a small incision in the patient's body (in the thoracic or abdominal wall);
introducing the guiding member by its distal portion inside the patient's body through said incision until the surface of the targeted anatomic organ, while blocking said incision with the fixation means so as to control the exchanges between the inside of the patient's body and the environment;
placing the base of the sucker on the surface of the targeted anatomic organ and applying a low negative pressure generated by the sucker of the guiding member on said surface by means of the negative pressure generator so as to stabilise the targeted anatomic organ;
with the targeted anatomic organ thus stabilised, passing a surgical instrument such as a robotic instrument, through the guiding member so that one of its ends protrudes outside the base of the guiding member and penetrates inside the targeted anatomic organ;
pursuing the surgical procedure inside the targeted anatomic organ by intervening with the surgical instrument.

The present invention is also related to the use of said guiding member and/or said method in cardiac surgery or in thoracic surgery.

As a second object, the present invention also concerns a surgical instrument adapted to cardiac surgery, and in particular to atrial defibrillation comprising insertion means for insertion inside the heart chamber, and cutting means connected at a connection zone to said insertion means, for creating lesions inside the heart chamber, said instrument being such that both its translation and rotation movements are controlled by a robotic system preferably coupled to a 3D-imaging system.

Preferably, the insertion means correspond to a rigid elongated stem delimited by an outer wall, with a main axis, and having a proximal end and a distal end, said proximal end being connected to the robotic system, while the distal end is free.

Preferably in said instrument, the cutting means comprise a flexible spreadable support structure with an inner surface and an outer surface, and an electrode mesh or network arranged on the outer surface of said support structure.

Preferably, said spreadable support structure corresponds to a dome structure having a tip and a base, said base being free and said tip being connected at the connection zone to the outer wall of the stem.

Advantageously, the instrument according to claim 12, wherein the dome structure is subdivided into dome sections able to selectively adopt a rest configuration for which all the dome sections are folded up along the outer wall of the stem, and a plurality of working configurations for which at least one dome section selectively spreads from the stem according to a spreading angle defined by the main axis of the stem, the connection zone and the base of the dome structure.

Preferably, the electrode mesh or network comprises a plurality of parallel electrodes arranged both radially and circularly on the outer surface of said dome structure, and activable selectively by the robotic system.

The present invention is also related to a method for performing an atrial defibrillation using the instrument according to the second object of the invention, said method comprising the following steps:
making a small incision in the thoracic wall of the patient so as to introduce guiding means inside the patient's cavity until the outer surface of the heart chamber, whereon said guiding means are placed;
stabilising said guiding means by attaching them to an immobile surface such as a surgical table;
under the control of the robotic system, passing the instrument through said guiding means by its distal end, with the dome structure in rest configuration, until said instrument reaches the heart chamber and penetrates inside the heart chamber;
positioning the instrument inside the heart chamber relatively to the atrial wall and following a predefined sequence of translation and rotation movements of the stem and of the dome structure corresponding to a sequence of working configurations for the dome structure;
coupling said sequence with a predefined activation sequence wherein different electrodes of the electrode network are selectively activated, so as to create selective lesions at precise locations in the atrial wall, said lesions being able to stop the electrical impulses associated to atrial fibrillation.

Preferably, in said method, a surgical protocol is pre-established by taking a series of 3D images of the heart with the 3D-imaging system and treating said images with the robotic system so as to predefine the sequence of rotations and translations to give to the instrument as well as the activation sequence of the electrodes in the electrode network.

Preferably, the 3D-imaging system coupled to the robotic system takes a series of 3D-images as a function of time, preferably in real time, thereby allowing a monitoring of the surgical procedure.

A third object of the present invention concerns a surgical instrument adapted for hepatic surgery and comprising insertion means for insertion inside a target organ, and heating means for coagulating specific tissue regions inside said target organ, said heating means being connected to said insertion means, said instrument (81) being capable of translation and rotation movements controlled by a robotic system preferably coupled to a 3D-imaging system and being adapted for intra-hepatic surgery.

Preferably, the insertion means correspond to a rigid elongated rod with a main axis, a centre of gravity, a proximal end and a distal end, said proximal end being connected to the robotic system, while the distal end is free.

According to one preferred embodiment, the heating means comprise at least (i) a secondary rigid rod articulated on the main rod via connection means and provided with a main axis, a proximal end and a distal end, and (ii) at least one electrode activable by the controlling means, preferably by radiofrequency.

According to a second preferred embodiment, the heating means comprise at least one bipolar electrode articulated on the main rod via connection means, said bipolar electrode preferably consisting of a first needle and a second needle, each of said needles being defined by a main axis, and being activable by the controlling means, preferably by a radiofrequency source.

Advantageously, in the first preferred embodiment, the instrument comprises two primary monopolar electrodes which are at least part of the secondary rod and are activable selectively by the controlling means, preferably by radiofrequency.

Preferably, in said first embodiment or in said second embodiment, the instrument further comprises at least one secondary monopolar electrode arranged at the distal end of the main rod and activable selectively from said primary electrodes or said bipolar electrode, respectively.

Moreover, in an advantageous manner, the main rod possesses six degrees of freedom, four of them being able to be blocked in operating conditions by the controlling means so as to allow only two degrees of freedom, one translation along and one rotation around its main axis.

Preferably, in the first embodiment, the secondary rod of the instrument has its main axis parallel to the main axis of the main rod and presents two degrees of freedom, one translation along and one rotation around its main axis so that the height and the distance of the secondary rod relatively to the main rod can be adjusted by the controlling means, the distance being adjustable at a value varying from O to a maximum value.

Preferably, in the second embodiment, the bipolar electrode of the instrument presents different degrees of freedom, one rotation around their main axis for each of said first and second needles and one translation along said axis so that the distance of the needles relatively to the main rod can be adjusted by the controlling means, said distance being adjustable at a value varying from O to a maximum value.

Advantageously, in the third object of the invention, the hepatic instrument is able to adopt one rest configuration and at least one working configuration, each of said configurations being defined by a different relative position of the insertion means and/or the heating means, the instrument being not functional in rest configuration but being functional in working configuration.

Preferably, when the instrument is in rest configuration, the secondary rod or the bipolar electrode is folded up inside the main rod (distance main rod/secondary rod or main rod/bipolar electrode equal to 0), and all the electrodes are unactivated.

Preferably, in the first preferred embodiment, when said instrument is in a working configuration, the secondary rod spreads out from the main rod, its main axis being parallel to the one of the main rod and distanced to it of a certain distance greater than 0 and at least of the electrode is activated.

Preferably, in the first preferred embodiment, when said instrument is in a working configuration, the bipolar electrode spreads out from the main rod, the main axis for each of said first and second needles being parallel to the main axis of the main rod and distanced to it of a certain distance greater than 0 and at least of the electrode is activated.

The present invention is also related to a method for coagulating an intra-hepatic tumour of a certain shape, using the hepatic instrument (instrument as third object of the invention), comprising the following steps:
making a small incision in the abdominal wall of the patient so as to introduce guiding means inside the patient's cavity until the outer surface of the liver, whereon said guiding means are placed;
stabilising said guiding means by attaching them to an immobile surface such as a surgical table;
under the control of the robotic system, passing the instrument through said guiding means by its distal end, with the instrument in rest configuration, until said instrument reaches the liver and penetrates inside the hepatic parenchyma;
positioning the instrument inside the hepatic parenchyma relatively to the hepatic wall and following a predefined sequence of translation and rotation movements of the main rod and the secondary rod corresponding to a sequence of working configurations;
coupling said sequence with a predefined activation sequence wherein different electrodes of the electrode network (first and second electrodes) are selectively activated, so as to lead to a tissue coagulation at precise target locations in the liver corresponding to tumour tissues.

Preferably in said method, a surgical protocol is pre-established by taking a series of 3D images of the liver and of the tumour with the 3D-imaging system and treating said images with the robotic system so as to predefine the sequence of rotations and translations to give to the instrument as well as the activation sequence of the electrodes in the electrode network.

Preferably in said method, the 3D-imaging system coupled to the robotic system takes a series of 3D-images as a function of time, preferably in real time, thereby allowing a monitoring of the surgical procedure.

A fourth object of the invention concerns a surgical assembly comprising one of the surgical instruments according to the invention (second or third object of the invention), and controlling means for controlling said surgical instrument.

Another object of the invention concerns a surgical assembly comprising a guiding member according to the invention and one of the surgical instruments according to the invention (second or third object of the invention).

Preferably, said surgical assembly further comprises controlling means for controlling said guiding member and said surgical instrument.

The present invention is also related to the use of the surgical instruments and/or the surgical assemblies according to the invention.

### SHORT DESCRIPTION OF THE DRAWINGS

FIG. 1 represents an overall view of a guiding member according to one preferred embodiment.

FIG. 2 represents a detailed view of FIG. 1 without stabilisation means.

FIG. 3 represents a detailed view of FIG. 3 without fixation means.

FIG. 4 represents a detailed underview of a guiding member according to the present invention.

FIG. 5 represents a guiding member according to the present invention as attached to a surgical table.

FIG. 6 represents a guiding member according to the invention as inserted in the thoracic or abdominal wall of the patient's body and fixed to a surgical table.

FIG. 7 illustrates a guiding member according to the present invention as positioned on the outer surface of an anatomic organ, with a surgical instrument passing through the guiding member and protruding inside the anatomic organ.

FIG. 8a-8b represent a first instrument according to one preferred embodiment of the invention, adapted for cardiac surgery, with its dome structure in rest configuration.

FIG. 9 represents a side view of the same instrument as in FIGS. 8a and 8b, but with its dome structure in a first working configuration.

FIG. 10a represents a face view of the instrument of FIG. 9.

FIG. 10b represents a face view of an instrument adapted for cardiac surgery according to another preferred embodiment of the invention.

FIG. 11a-11b represent a side view of an instrument according to one preferred embodiment of the invention, with its dome structure in a second working configuration and in a third working configuration, respectively.

FIG. 12a illustrates the positioning of guiding means on the outer surface of the heart chamber during the surgical procedure.

FIG. 12b-12d show an instrument according to the invention as placed inside the heart chamber in a first, second and third working configuration respectively, during the surgical procedure.

FIG. 13 is a cross view of the multi-layer dome structure in an instrument according to one preferred embodiment of the invention.

FIG. 14 shows an overall view of a whole surgical assembly adapted for cardiac surgery according to the present invention.

FIG. 15 shows the actuators' locations in a surgical assembly adapted for cardiac surgery according to the present invention.

FIG. 16a represents a second instrument of the present invention, adapted for hepatic surgery, according to one preferred embodiment, with its main rod and its secondary rod.

FIG. 16b represents an instrument adapted for hepatic surgery, according to another preferred embodiment of the present invention.

FIG. 17 shows the different degrees of freedom of the instrument according to the present invention.

FIG. 18 illustrates the positioning in a patient of a guiding member on the outer surface of its liver, during a surgical procedure using the instrument adapted for hepatic surgery according to the present invention in working configuration.

FIG. 19 represents a surgical assembly according to the present invention with the instrument adapted for hepatic surgery positioned in the hepatic parenchyma.

FIG. 20 illustrates the positioning of the guiding member on the outer surface of the liver during the surgical procedure.

FIG. 21 illustrates how a surgical assembly comprising an instrument adapted for hepatic surgery according to the present invention is linked to a surgical table for stabilisation purposes.

FIG. 22 illustrates the connection of said surgical assembly to a robotic system and to a 3D-imaging system according to the present invention.

### DETAILED DESCRIPTION OF THE INVENTION

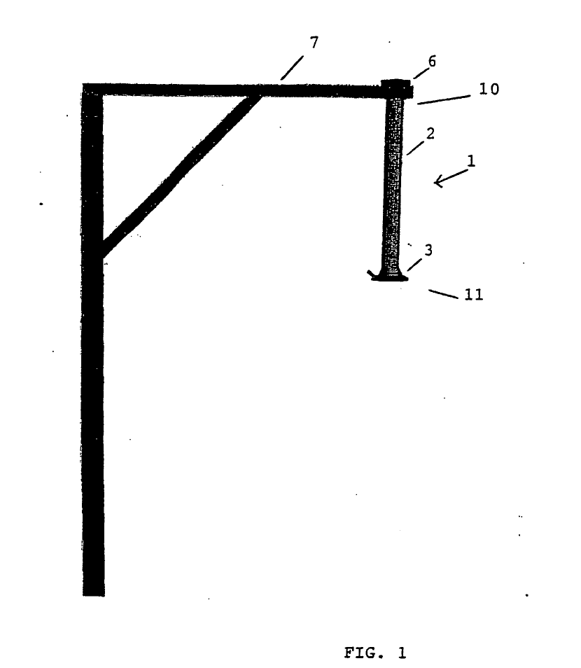

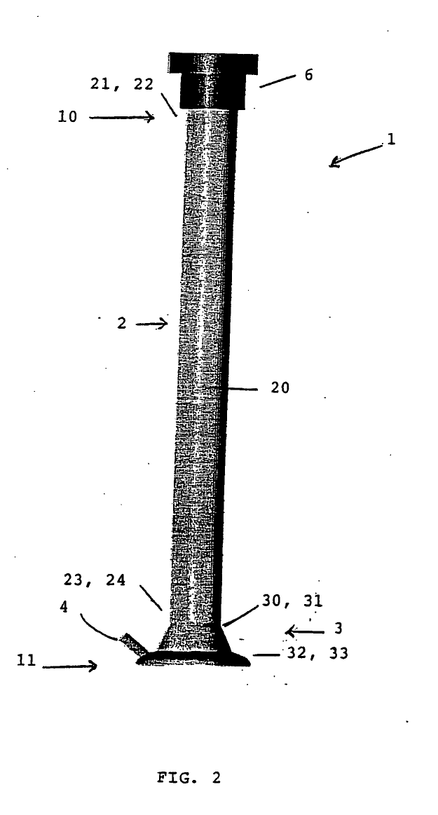

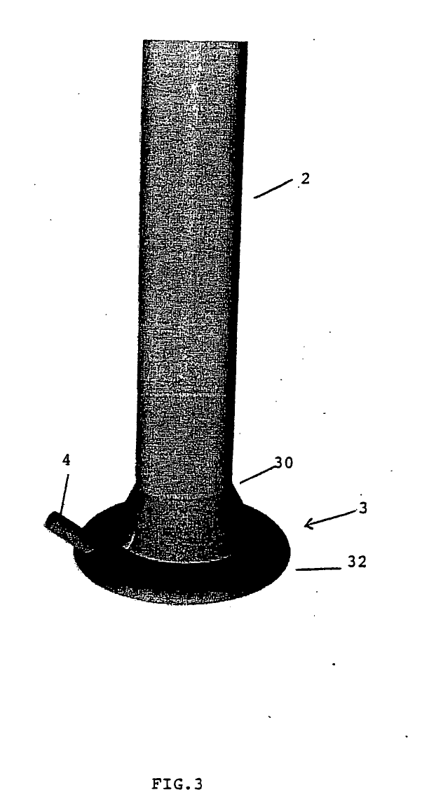

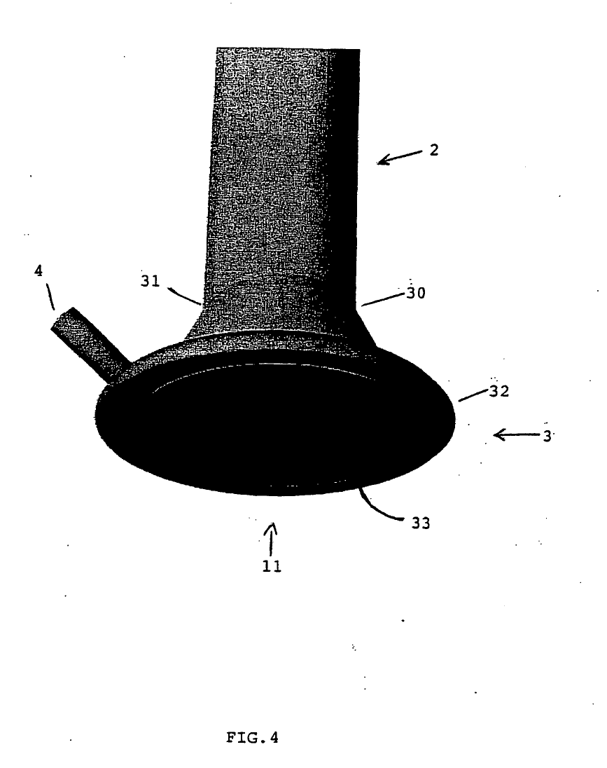

As illustrated in FIGS. 1 and 2, the guiding member 1 according to the present invention comprises an elongated rigid body 2, preferably tubular, having an inner lumen 20. Said body 2 presents a proximal end 21 with a first open tip 22 and a distal end 23 with a second open tip 24.

The guiding member 1 according to the invention also comprises sealing means corresponding to a sucker 3 having a first end or top 30 with a first open tip 31 and a second end or base 32 with a second open tip 33. The sucker 3 is preferably of conical shape, with its base 32 being larger than its top 30.

In the guiding member 1, the sucker 3 is mounted on the body 2, with its top 30 connected to the distal end 23 of the body 2 and in such a manner that communication inside the guiding member is allowed from the first open tip 22 of the body 2 to the second open tip 33 of the sucker 3.

The configuration, in particular the dimensions, of the ensemble body 2/sucker 3 is such that a surgical instrument may pass through it.

For the guiding member so configured, a proximal portion 10 and a distal portion 11 are defined.

Furthermore, the sucker 3 is provided with connection means 4 for connecting it to an external negative pressure generator 5.

It should be noted that the composition of the different elements constituting the guiding member 1 should be chosen in biocompatible materials.

Moreover, the material for the body 2 should be selected for providing to the guiding member 1 a rigidity sufficient for safely and precisely guiding a robotic surgical instrument to its target (i.e. an anatomic organ) that is to say without any risk of deviation for the instrument. In addition, the body is advantageously in a transparent material so that the surgeon may see inside, for example by means of an endoscopic camera when the guiding member 1 is placed into the patient's body.

Concerning the material of the sucker 3, it has to provide sufficient flexibility so that the sucker 3, when the guiding member 1 is used, may adapt its base 32 to the surface of the targeted anatomic organ (preferably a heart or a liver) and ensure thereby its sealing function in regard to the targeted anatomic organ, without any risk of bleeding within the patient's body.

The guiding member 1 further comprises, at the proximal end 21 of the body 2, fixation means connected to stabilisation means for sufficiently stabilising the targeted anatomic organ, when the guiding member is used, and thereby contributing to the safe working of the surgical instrument.

Preferably, said fixation means correspond to a trocar 6 as commonly used by the man skilled in the art.

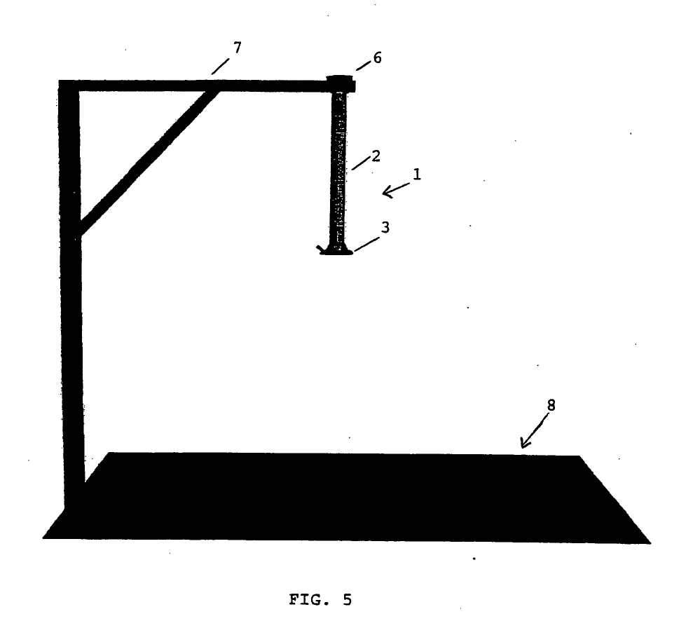

Preferably, said stabilisation means correspond to immobile support means consisting in at least one support arm 7 connectable to a surgical table 8, as illustrated in FIG. 5.

Preferably, the support arm is articulated so that it is possible to adjust the distance of the guiding member to the patient's body.

It should be noted that in the guiding member 1, the body 2 may be provided with a valve disposed in its inner lumen, said valve being specially useful to avoid the blood to flow through the body 2, in the case of surgeries when the blood is under high pressure (a cardiac cavity, for example).

Concretely, the method for performing a surgical intervention on a beating heart or a liver using a surgical instrument, preferably a microrobotic surgical instrument, coupled to the guiding member according to the present invention, comprises the following steps:
connecting the sucker 3 of the guiding member 1 to an external negative pressure generator 5;
creating a small incision in the patient's body (in the thoracic wall 80 or abdominal wall 80′);
introducing the guiding member 1 by its distal portion 11 inside the patient's body through said incision until the surface of the targeted anatomic organ 200, while blocking said incision with fixation means 6 such as a trocar 6 so as to control the exchanges between the inside of the patient's body and the environment;
placing the base 32 of the sucker 3 on the surface of the targeted anatomic organ 200 and applying a low negative pressure generated on said surface via the sucker 3 of the guiding member 1, by means of the negative pressure generator 5 so as to firmly stabilise the targeted anatomic organ 200;
attaching the guiding member 1 with the trocar 6 to a support arm 7 and to a surgical table 8;
with the targeted anatomic organ 200 thus stabilised, passing a surgical instrument 9 such as a robotic instrument, through the guiding member 1 so that one of its ends 90 protrudes outside the base 32 of the guiding member 1 and penetrates inside the targeted anatomic organ;
pursuing the surgical procedure inside the targeted anatomic organ by intervening with the surgical instrument 9.

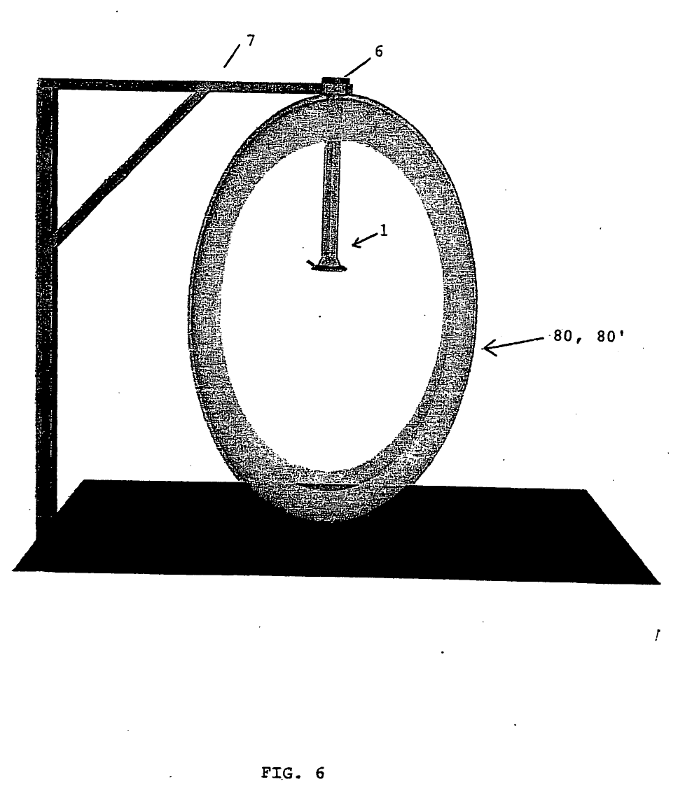

The guiding member 1 as inserted in the thoracic wall 80 or abdominal wall 80′ of the patient's body and fixed to a surgical table is illustrated on FIG. 6.

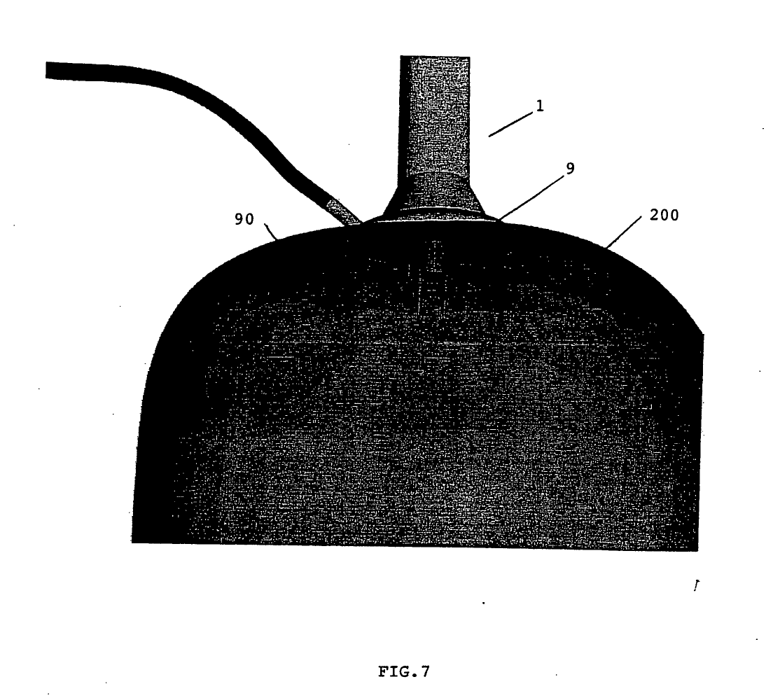

FIG. 7 illustrates the guiding member 1 as positioned on the outer surface of the anatomic organ 200, with the surgical instrument 9 passing through the guiding member and protruding inside the anatomic organ 200.

Once the intervention with the surgical instrument is finished, the surgical procedure is ended by:
removing the surgical instrument 9 outside the targeted anatomic organ and outside the guiding member 1;
stopping the working of the generator 5;
removing the guiding member 1 outside the patient's body by the incision made initially;
closing said incision.

Comparatively to the techniques of the state of the art, the technique using the guiding member according to the present invention offers several advantages.

A main advantage is that said guiding member allows a surgical procedure as less invasive as possible, through the stabilisation of the targeted anatomic organ. Due to said stabilisation, the use of a robotic surgical instrument can be considered.

Another advantage of the guiding member according to the present invention is that it does not penetrate inside the targeted anatomic organ so that the manipulation of the surgical instrument inside the targeted anatomic organ is facilitated. Indeed, the movements of the surgical instrument inside said organ are not restricted by the presence of the guiding member, so the number of freedom degrees is maximised and the accessibility of the internal volume of the anatomical organ is optimised.

The guiding member according to the invention further offers the advantage of being safe and secured for the patient.

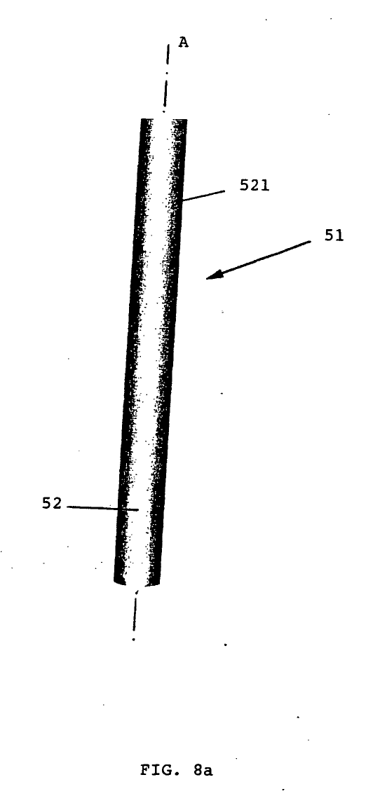

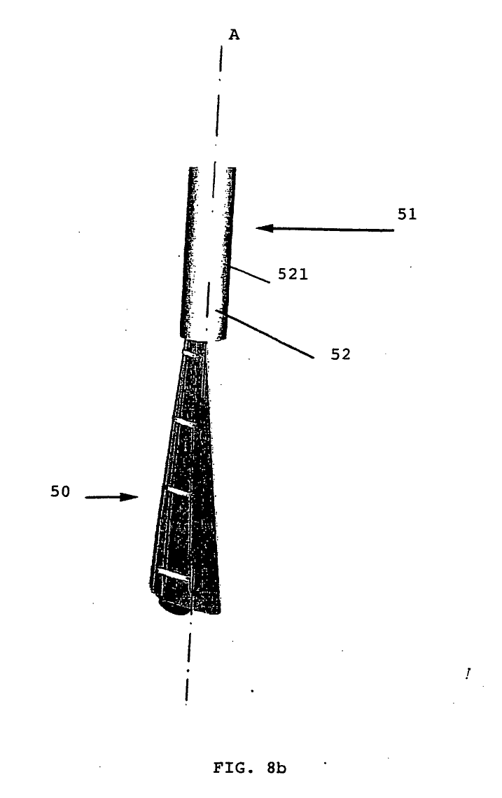

FIGS. 8a to 15 are related to a first instrument according to the present invention, which is specifically adapted for cardiac surgery, and more particularly for atrial defibrillation.

As illustrated in FIG. 8a-8b, said instrument 51 according to the present invention comprises insertion means which preferably take the form of a rigid rod 52 of cylindrical shape with a main axis A around which the stem 52 is able to rotate, an inner wall 520 and an outer wall 521.

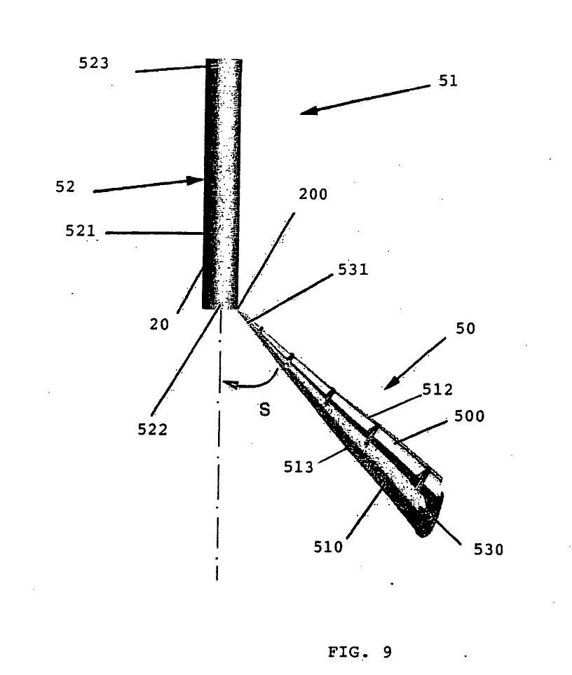

The rod 52 presents a proximal end 523 and a distal end 522 as shown in FIG. 9, the distal end 522 being free and the proximal end 523 being connected to controlling means, more precisely, to a robotic system 300 including a robotic arm 700 and preferably coupled to a 3D-imaging system 400 (see hereafter). The rod 52 can be moved in rotation and in translation and said robotic system 300 controls via the robotic arm 700 with a near millimetre precision the movements of the rod 52 i.e. its translation and rotation movements.

As illustrated in FIG. 8b and FIG. 9, along its outer wall 521, the rod 52 is connected to heating and cutting means 50, said heating and cutting means 50 comprising cutting elements 500 supported by support means 510.

Said support means 510 correspond to a spreadable and orientable structure controlled by the robotic system 300. Said support means 510 preferably take the form of a flexible dome structure similar to the one of an umbrella with a base 530 and a tip 531, an outer surface 512 and an inner surface 513.

Said dome structure 510 is movably affixed to the outer wall 521 of the rod 52 by its tip 531 at a connection zone 540, said dome structure 510 being able to be translated along and rotated around the main axis A of the rod 52 under the control of the robotic system 300, while the base 530 of the dome structure 510 is free.

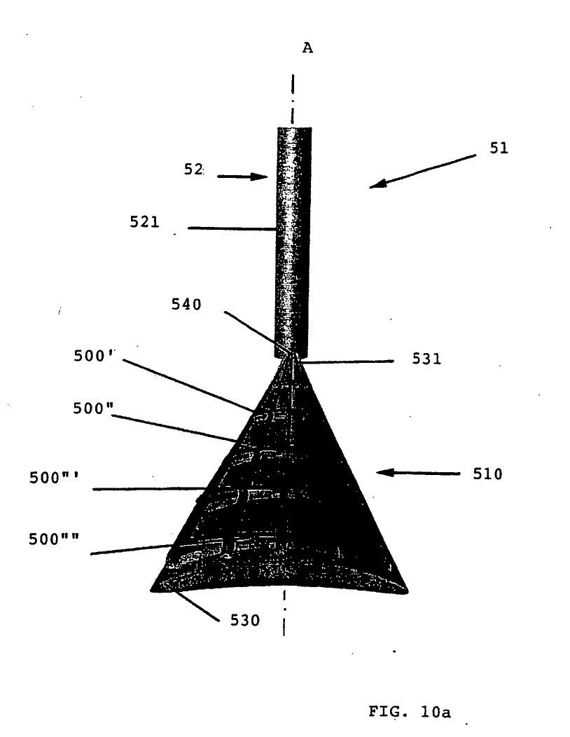

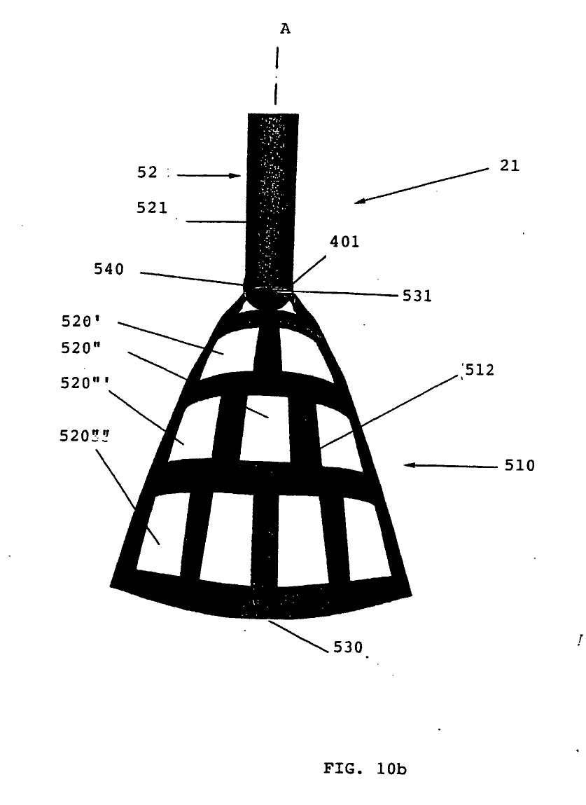

In one preferred embodiment of the present invention, the connection zone 540 is constituted by a spherical joint 401 (see FIG. 10b).

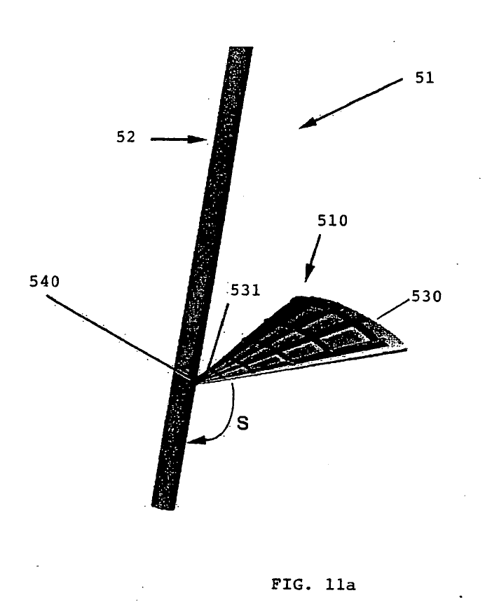

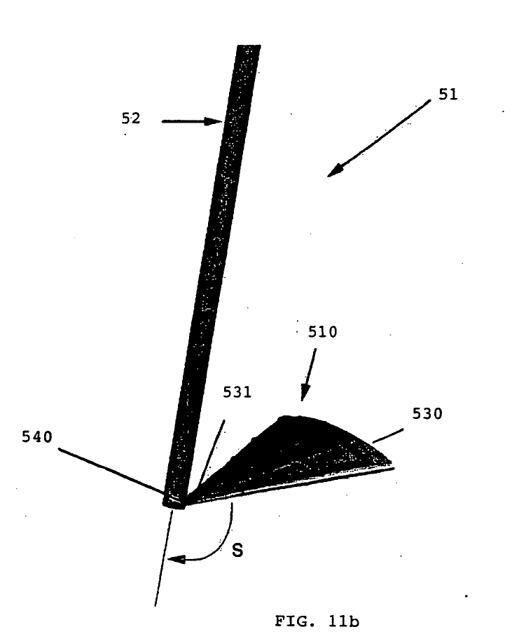

Furthermore, as illustrated in FIGS. 11a and 11b, the dome structure 510 is deployable out according to a selectable spreading angle S from said connection zone 540, under the control of the robotic system 300.

For said dome structure 510, one defines (i) a rest configuration as illustrated in FIG. 8a, wherein the dome structure is closed with the dome structure 510 folded up along the outer wall 521 of the rod 52 and (ii) several working configurations as illustrated in FIGS. 9 to 13, wherein the dome structure is spread out from the rigid stem 52 with a variable angle.

Preferably, the dome structure 510 is subdivided into dome sections able to selectively adopt a rest configuration for which all the dome sections are folded up along the outer wall 521 of the stem 522 and a plurality of working configurations for which at least one dome section selectively spreads from the stem 52 according to a spreading angle S defined by the main axis A of the stem 52, the connection zone 540 and the base 530 of the dome structure 510. Examples of such embodiments are given by FIGS. 9-11b.

The controlling means and robotic system are able to control the movements of the dome structure i.e. its rotation and translation movements as well as its opening(spreading)/closing movements, preferably via actuators and micro-actuators. Said actuators and micro-actuators can be of several types, including electrostatic, magnetic, piezo-electric, thermic, shape memory alloy (SMA), fluidic and electro-rheologic types.

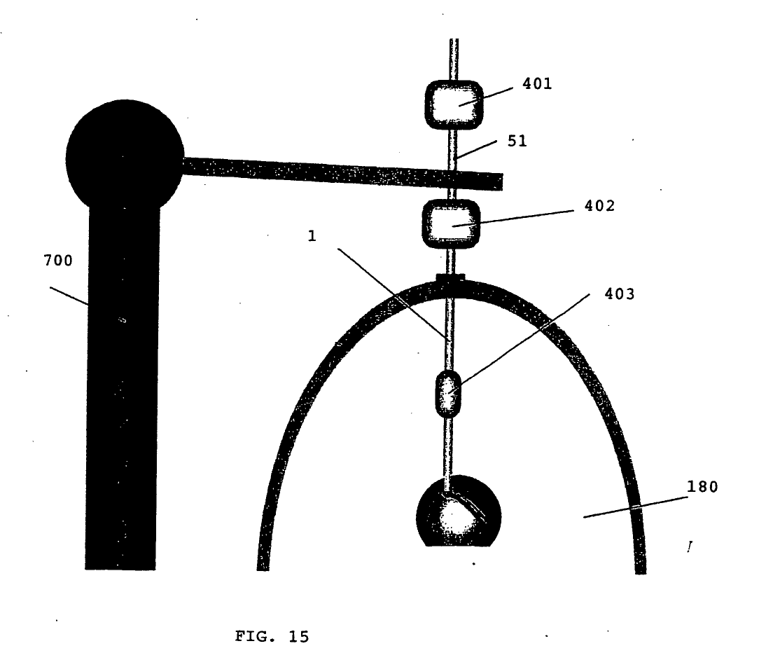

Said actuators and micro-actuators may have different positions (see hereafter). For example, as shown in FIG. 15, actuators can be placed on the instrument 51 at position 401 or 402, or on the guiding member 1 at position 403.

The cutting elements 500 of the heating and cutting means 50 take the form of a series of electrodes 500′,500″,500′″ . . . able to transmit radio frequency (RF) and which can be activated independently from each other through the computer of the robotic system 300. The cutting elements 500′,500″,500′″ . . . are more precisely organised as a mesh or network of electrodes covering at least part of the outer surface 512 of the dome structure 510. Preferably, this mesh or network comprises a plurality of parallel electrodes arranged radially and circularly on the outer surface 512 of the dome structure 510.

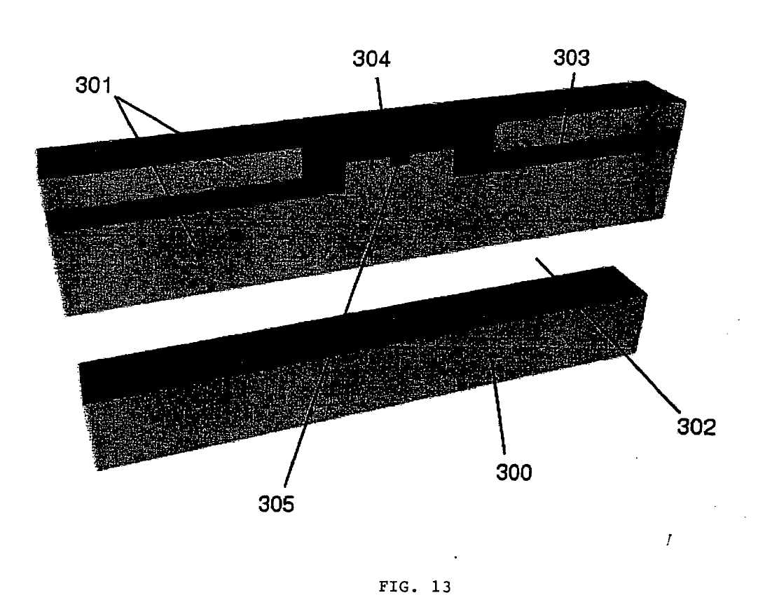

In one preferred embodiment of the invention illustrated in FIGS. 10c and 13, the dome structure 510 comprises two flexible layers 501 and 503 of PDMS (polydimethylsiloxane), with an inner empty space 502 between them. This space 502 can be filled by a fluidic actuator, preferably a sterile saline solution, so as to move the dome structure 510, and consequently the electrodes 500′,500″,500′″ . . . , in operating conditions.

The saline solution can also be used to cool the electrodes 500′,500″,500′″ . . . through small canals, placed near these electrodes, allowing a communication between the inner space 502 and the outer surface 512 of the dome structure 510. The cooling effect is obtained as the cool saline solution passes from the inner space 502 to the outer surface 512 of the dome structure 510 near the RF heated electrodes 500′,500″,500′″.

In said embodiment, the electrodes 500′,500″,500′″ . . . may take the form of continuous metallic strips 303 inlayed in the layer 502 of PDMS. These metallic strips 303 are isolated from the surface 512 of the dome structure 510 by the PDMS itself, except in some raised regions 304, where they constitute the visible portions of the electrodes 500′,500″,500′″ . . . . Just below the raised regions 304 of the metallic strips 303, can be placed some special sensors 305, like temperature sensors or strength gauges, for example.

In another preferred embodiment of the present invention illustrated in FIG. 10b, the spaces between the cutting elements 500′,500″,500′″ . . . , in the dome structure 510, are empty so as to define holes 520′,520″,520′″ . . . through which the blood may flow when the instrument is introduced inside the patient.

Generally, the composition and dimensions of the rod 52 as well as the one of the cutting means 50 are compatible with their technical use (in particular, in terms of biocompatibility) and can be easily adapted from the present description by the man skilled in the art.

Concretely, the instrument 51 according to the present invention can be used as follows in order to perform tissue ablation on a beating heart suffering from atrial fibrillation.

Firstly, the 3D-imaging system 400 takes a series of 3D-images from the heart and said 3D-images are recorded, treated and analysed by the computer of the robotic system 300 in order to establish the operating protocol to be performed that is to say the sequence of movements (translations+rotations) to order to the instrument 51 (rod 52+dome structure 510) as well as the sequence for activating the different 500′,500″,500′″ of the cutting means 50.

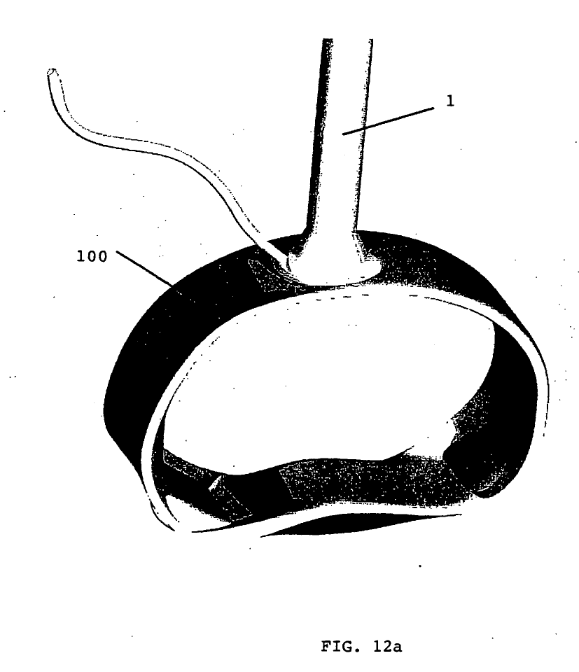

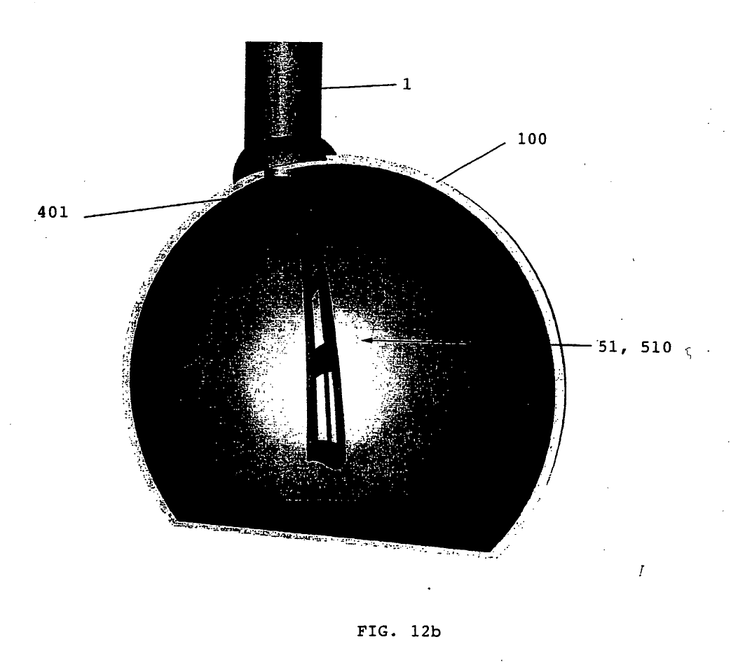

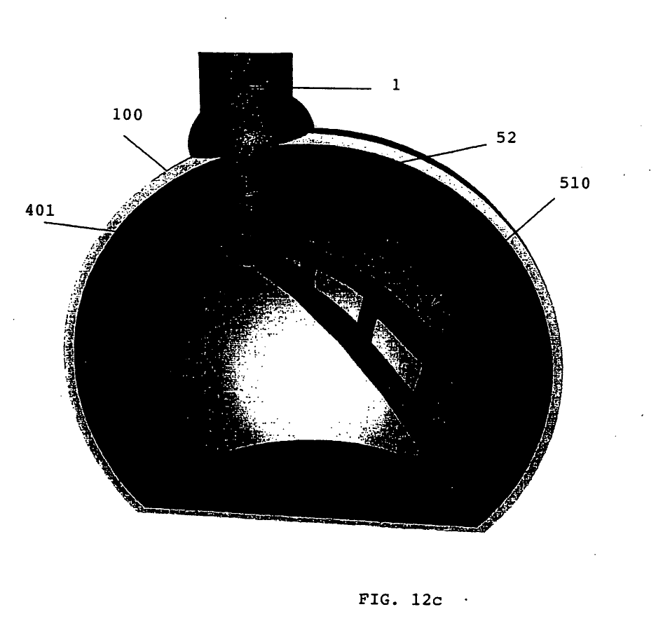

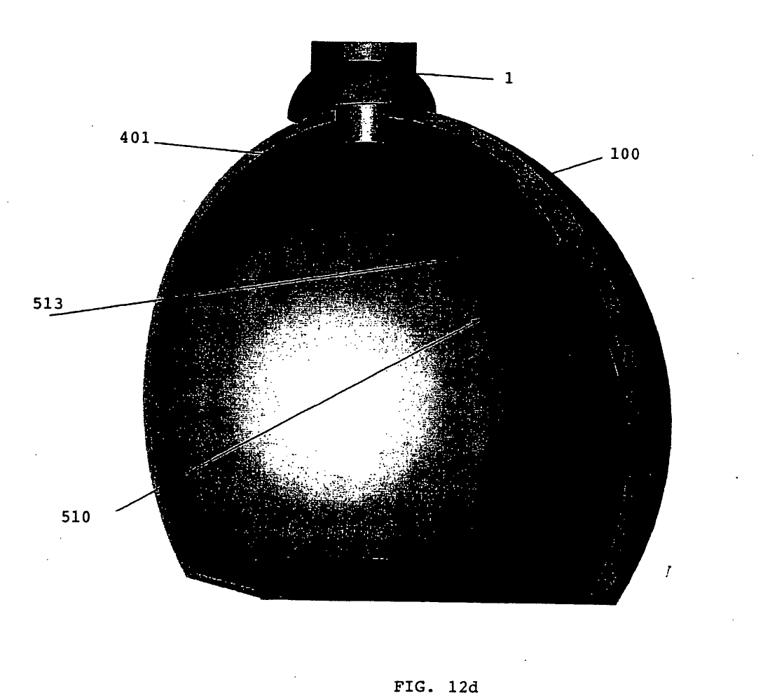

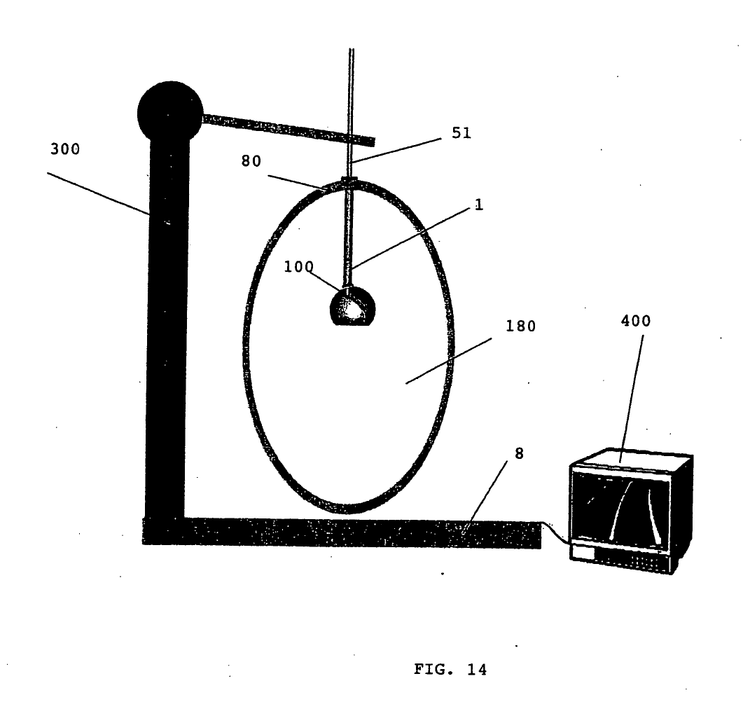

Once said protocol has been established, the surgeon makes a fine incision in the patient's thoracic wall 80 on a predetermined location through which guiding means 1 having an inner lumen are introduced into the patient's body (thoracic cavity 180) and placed on the outer surface of the heart chamber 100 (right or left atrium; see FIG. 12a). Said guiding means is preferably the guiding member of the present invention other guiding means can also be used. Said guiding means 1 are connected to a rigid support 8, for example a surgical table (see FIG. 14).

The surgeon then activates the robotic system 300 so as to perform the surgical procedure and monitors at every time said procedure through the 3D-imaging system 400.

The robotic system 300, following the predetermined sequence of movements mentioned hereabove, introduces the instrument 51 according to the present invention into the inner lumen of the guiding means 1 in place, with the dome structure 510 in rest configuration. The instrument 51 penetrates inside the heart chamber 100 at a predetermined location, where it undergoes a sequence of configurational changes into different working configurations and with specific electrodes 500′,500″,500′″, . . . in the electrode network being activated so as to create precise incisions inside the heart (cutting done by heating), said incisions being capable of stopping electrical impulses associated to atrial fibrillation (see FIG. 12b, FIG. 12c and FIG. 12d).

It should be noted that the robotic system 300 is provided with securing means activable in case of abnormalities for interrupting the working of the robotic system 300 so that the surgeon may continue manually the surgical procedure.

In this manner, the instrument of the present invention offers all the guarantees of security for the patient.

Moreover, with the instrument and method according to the invention, the time of the surgical procedure is reduced to its minimum as it is optimised through the integration of a maximum of robotic surgical steps.

Moreover, as the instrument and method according to the invention use the robotic system coupled to the 3D-imaging system and no exposition of the patient to X-Rays, they are safer for the patient than the methods and instruments of the state of the art.

In addition, as only 1 cm large incisions in the patient's thoracic wall is necessary for introducing the instrument inside the patient's body and in the heart chamber, the traumatism induced by the method using the instrument according to the invention is minimised for the patient.

Furthermore, the composition and the configuration of the instrument according to the invention is such that the combination of the movements of the rod and the ones of the dome structure allows a tight contact between the electrode mesh and the atrial internal wall. In other words, a further advantage of the present invention is that these movements ensure a good flexibility of the instrument which may adapt the electrode mesh to any atrial wall portion, thereby allowing incisions with near a millimetric precision, even in regions hardly accessible.

As illustrated hereabove, the instrument and method according to the present invention thus offer undeniable advantages over the state of the art.

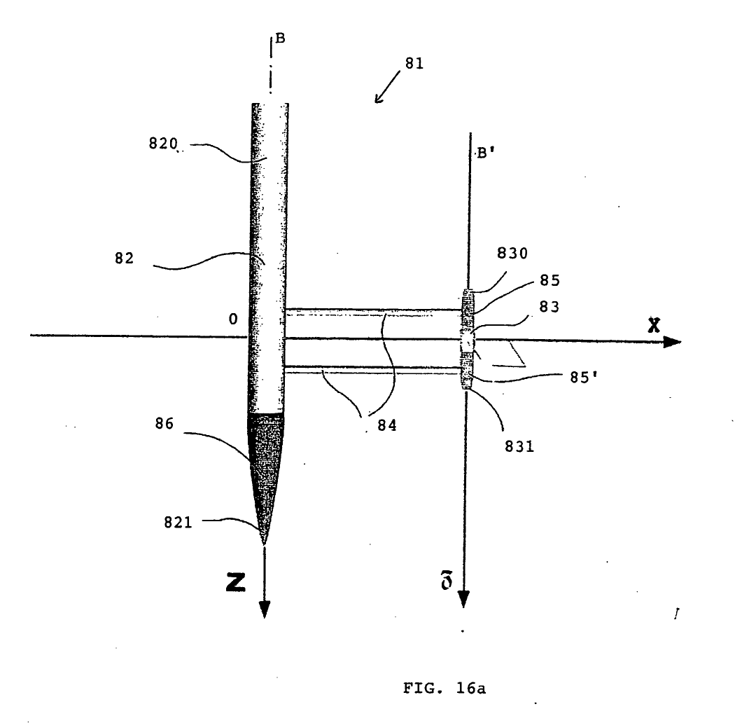

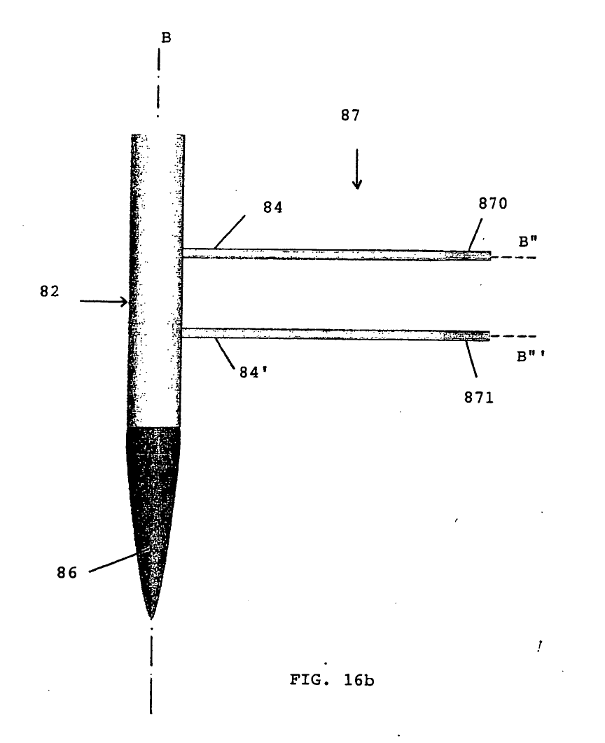

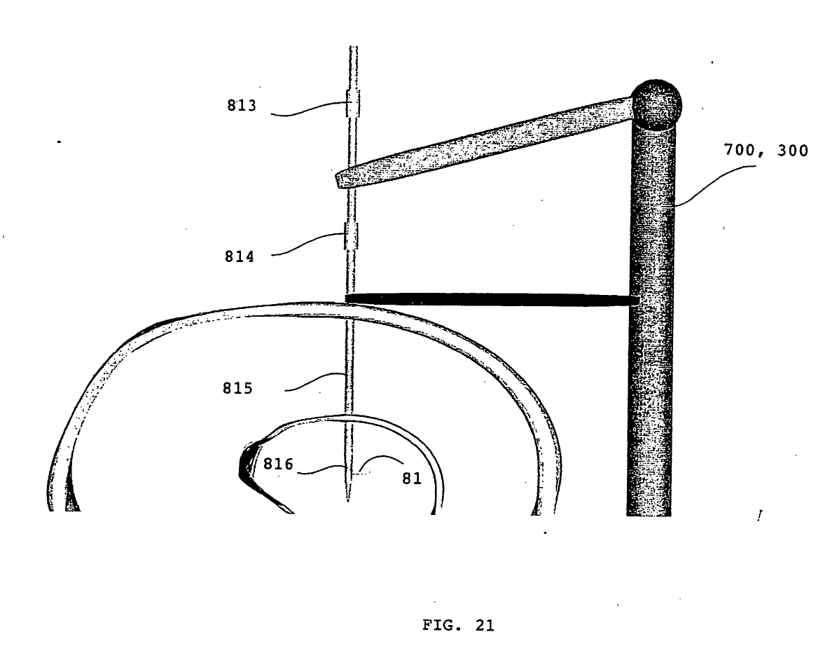

Another aspect of the present invention concerns a second surgical instrument, preferably specifically adapted for hepatic surgery. FIGS. 16a to 22 illustrate this aspect of the invention.

FIG. 16a represents an instrument adapted for hepatic surgery according to one preferred embodiment of the invention. Said instrument, with the reference 81, comprises a main rigid rod 82 and a secondary rod 83, both of substantially cylindrical shape, the secondary rod 83 being articulated on the main rod 82 via one or more connection arms 84.

Each of the main rod 82 and the secondary rod 83 has a proximal end 820 or 830 respectively and a distal end 821 or 831 respectively.

The proximal end 820 of the main rod 82 is connected to a controlling system corresponding to a robotic system including a robotic arm 700 and preferably coupled to a 3D-imaging system 400, while the distal end 821 of said main rod 82 is free and is conformed as a tip so as to easily penetrate inside the target organ i.e. the liver.

Similarly, the distal end 831 of the secondary rod 83 is also conformed as a tip.

In the instrument according to the invention, different degrees of freedom are associated to the main rod 82 as well as to the secondary rod 83.

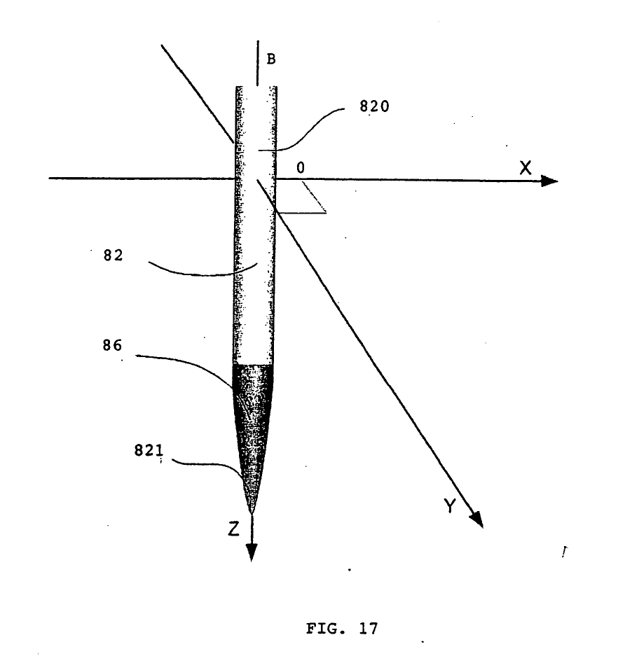

The main rod 82 comprises six degrees of freedom. If one defines a referential system (O,X,Y,Z) as illustrated in FIG. 17, with the Z axis corresponding to the main axis B of the rod 82 and the origin O (0,0,0) of said system corresponding to the centre of gravity of the rod 82, said six degrees of freedom are the followings:
three degrees of rotation around each of the axis X, Y, and Z;
and three degrees of translation along said axis X, Y and Z.

However, it should be noted that in operating conditions, when the instrument 81 is introduced inside the hepatic parenchyma, the main rod 82 has only two degrees of freedoms, the rotation and the translation along its main axis B (Z axis), the other degrees of freedom being blocked by the controlling means.

The secondary rod 83 has two degrees of freedom. The first degree of freedom corresponds to a translation along its main axis B′ so that an adjustment of the height of the secondary rod 83 relatively to the main rod 82 can be made via the controlling means. The second degree of freedom corresponds to a translation along the axis x perpendicular to the axis B′ so that an adjustment of the distance between the main rod 82 and the secondary rod 83 can be made via the controlling means.

The relative configuration of the main rod 82 and the secondary rod 83 is such that the main axis B′ of the rod 83 is always parallel to the main axis B of the rod 82.

The main rod 82 as well as the secondary rod 83 are thus able, under the control of the robotic system to be rotated and translated to a variety of accessible positions with a near millimetre precision as defined by their degrees of freedom mentioned hereabove.

Moreover, the secondary rod 83, as shown in FIG. 16a, is provided with (comprises) two primary electrodes 85,85′ respectively, which correspond to monopolar electrodes located at its proximal end 830 and at its distal end 831, respectively. The monopolar electrodes 85,85′ are activable, preferably selectively (i.e. separately) according to a sequence of activation (see below), via a radiofrequency generator under the control of the controlling means (robotic system).

Similarly, the main rod 82, as shown in FIG. 16a, is provided with (comprises) a secondary electrode 86, which corresponds to a monopolar electrode located at its distal end 821. The monopolar electrode 86 is activable, preferably via a radiofrequency generator under the control of the controlling means.

For this purpose, the primary electrodes 85,85′ and the secondary electrode 86 are linked to a source of radiofrequencies (radiofrequency generator), the working of which is under the control of the robotic system.

Other embodiments wherein the secondary rod 83 of the instrument comprises only one or more than two primary electrodes, and/or wherein the main rod 82 of said instrument comprises no electrode or more than one secondary electrode, are also parts of the present invention.

It should be noted that, by convention, the main rod 82 should be considered as insertion means, while the secondary rod 83, the primary electrodes 85,85′ and the secondary electrode 86 form the heating means of the instrument 81 according to the present invention. Moreover, the primary electrodes 85,85′ and the secondary electrode 86 form together an electrode network.

In addition, in the present invention, the instrument 81 may adopt different configurations: a rest configuration and a series of working configurations.

In the rest configuration, which corresponds to a non-working state of the instrument according to the invention, the primary electrodes 85,85′ and the secondary electrode 86 are switched off, and the secondary rod 83 is folded up inside the main rod 82 as shown in FIG. 17 (distance rod 82/rod 83 equal to zero with main axis B′ of rod 83 coinciding with main axis B of rod 82).

As illustrated in FIG. 16a, a working configuration is characterised by:
the secondary rod 83 spreading out from the main rod 82, (distance main rod 82/secondary rod 83 non equal to 0);
at least one of the primary electrodes 85,85′ is activated (switched on);
the secondary electrode 86 of the main rod 82 is activated or not (switched on or off).

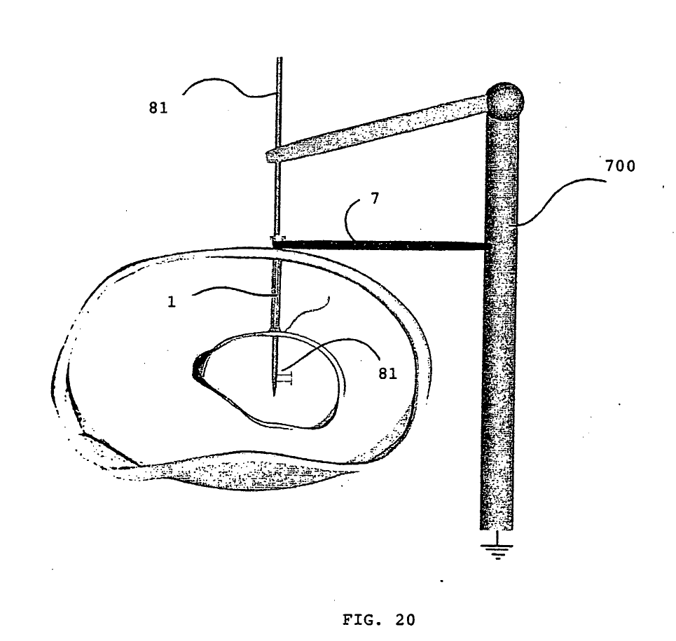

Actuators of the movements of the rod 82 and rod 83 may be part of the instrument 81 or of the surgical assembly. Four different locations for said actuators are represented in FIG. 20 and correspond to the references 813,814,815 and 816. Positions 815 and 816 correspond to two positions of an actuator on the instrument 81 itself, while positions 813 and 814 correspond to two different locations of said actuator somewhere else on a surgical assembly according to the invention, and more precisely on guiding means 1 which are used to introduce the instrument 81 inside the hepatic parenchyma.

The composition and dimensions of the main rod 82, the secondary rod 83, the primary electrodes 85,85′ and the secondary electrode 86 are compatible with their technical use (intra-hepatic surgery), in particular in terms of biocompatibility, and can be easily adapted from the present description by the man skilled in the art.

The actuators for moving the instrument 81 can be of several types including electrostatic, magnetic, piezo-electrical, thermic, shape memory alloy (SMA), fluidic and electro-rheologic actuators.

FIG. 16b represents another embodiment of the instrument according to the invention. In said embodiment, there is no secondary rod on the contrary to the first embodiment shown in FIG. 16a, but the instrument 81 comprises a bipolar electrode 87 consisting of a first needle 870 forming the first pole of the electrode 87 and a second needle 871 forming the second pole of the electrode 87. The secondary electrode 86 of the main rod 82 and the bipolar electrode 87 form together the electrode network of the instrument 81 according to said embodiment. Preferably, said needles 870,871 are connected to the main rod 82 via two different connection arms 84,84′. In said embodiment, the needles 870,871 have only one degree of freedom each, which corresponds to a translation along their main axis B″ and B′″ respectively. The translation along the axis B″ and B′″ are controlled by the controlling means so that the distance between each needle 870,871 and the main rod 82 can be adjusted. However in said embodiment, on the contrary to the embodiment shown in FIG. 16a, the height of the electrode 87 relatively to the main rod 82 can not be adjusted. The needles 870 and 871 of the bipolar electrode are such that they always remain parallel to each other.

Concretely, the instrument 81 according to the present invention can be used as follows in order to perform tissue coagulation on a working liver suffering from cancerous tumour.

Firstly, the 3D-imaging system 400 takes a series of 3D-images of the liver and of the tumour located therein, and said 3D-images are recorded, treated and analysed by the robotic system 300 (computer included in said robotic system) in order to establish, on the basis of said analysis (tumour shape and size), the operating protocol to be performed that is to say the sequence of movements (translations+rotations) to order to the instrument 81 (rod 82+rod 83) as well as the sequence for activating the different electrodes that is to say the primary electrodes 85,85′ and the secondary electrode 86 in the embodiment of FIG. 16a, or bipolar electrode 87 and secondary electrode 86 in the embodiment of FIG. 16b.

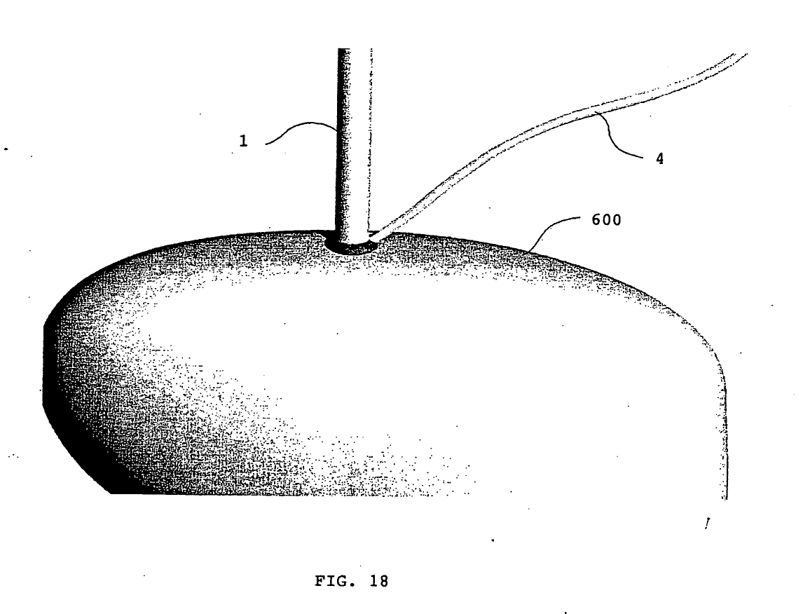

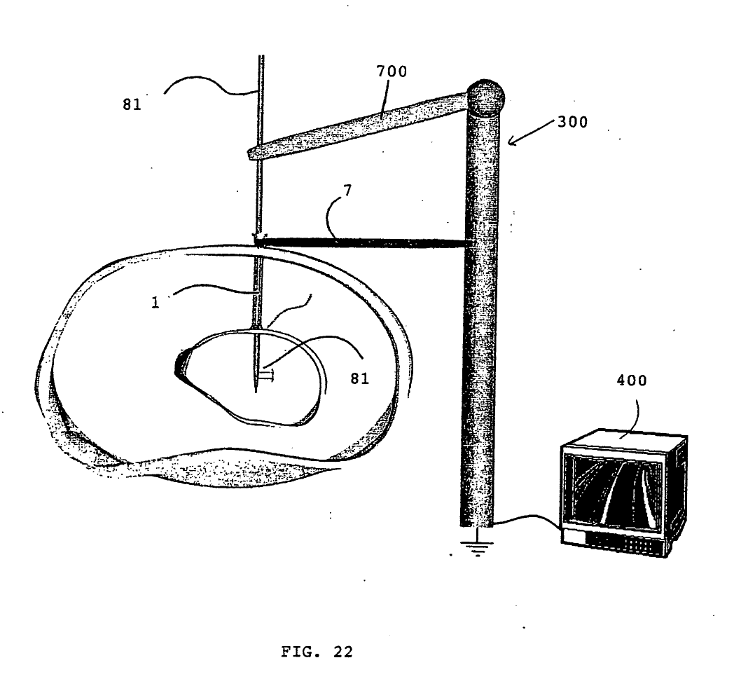

Once said protocol has been established, the surgeon makes a fine incision in the patient's abdominal wall 80′ (see FIGS. 20 and 22) on a predetermined location through which guiding means having an inner lumen are introduced into the patient's body (patient's cavity) and placed on the outer surface of the liver 600 (see FIG. 18). Preferably said guiding means correspond to the guiding member 1 according to the invention. Said guiding member 1 is connected via a rigid support arm 7 to a rigid support 8, for example a surgical table, and via connection means 4 to an external negative pressure generator (for stabilising the liver).

The surgeon then activates the robotic system 300 so as to perform the surgical procedure, and monitors at every time said procedure through the 3D-imaging system 400.

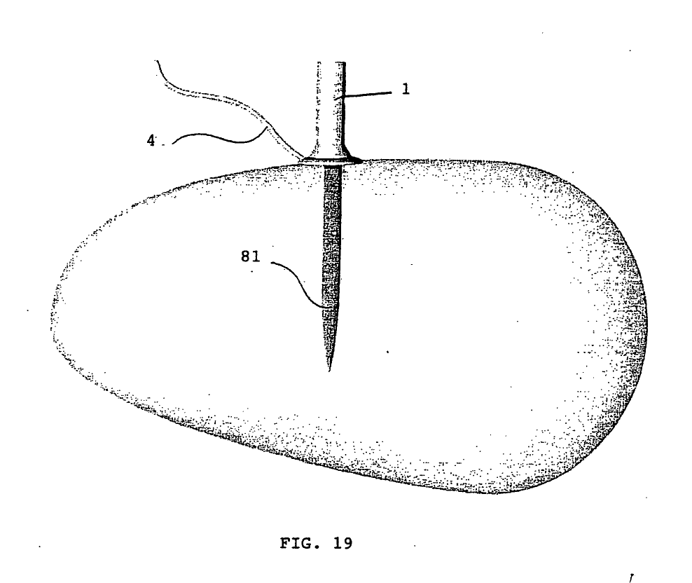

The robotic system 300, following the predetermined sequence of movements mentioned hereabove, introduces the instrument 81 according to the present invention into the inner lumen of the guiding means 1 in place, with the instrument 81 in rest configuration. The instrument 81 penetrates inside the hepatic parenchyma at a predetermined location to the tumour (see FIG. 19), where it undergoes a sequence of configurational changes into different working configurations as defined hereabove and with specific electrodes 85,85′,86 (in the embodiment of FIG. 16a) or 87,86 (in the embodiment of FIG. 16b) activated in the electrode network so as to heat precise regions inside the hepatic parenchyma (tumour regions), forcing said regions to coagulate.

It should be noted that the robotic system 300 is provided with securing means activable in case of abnormalities for interrupting the working of the robotic system so that the surgeon may continue manually the surgical procedure.

In this manner, the instrument 81 of the present invention offers all the guarantees of security for the patient.

Moreover, as only one incision in the patient's abdominal wall 80′ is necessary for introducing the instrument inside the patient's body and in the liver, the traumatism induced by the method using the instrument according to the invention is minimised for the patient.

Furthermore, the combination of the movement of the main rod 82 and of the secondary rod 83 in one embodiment or of the main rod 82 and of the bipolar electrode in another embodiment of the invention ensures a satisfying coagulation of only the target tissue volume heated by the electrodes with a near millimetre precision, while preserving the surrounding tissues.

In addition, the movement of the secondary rod in the first embodiment of the invention allows to reach hepatic regions even when these regions are hidden behind blood vessels.

As illustrated hereabove, the instrument and method according to the present invention thus offer undeniable advantages over the state of the art.

### NUMERICAL REFERENCES

### Guiding Member

1: guiding member
2: rigid body
20: inner lumen of rigid body 2
21: proximal end
22: first open tip
23: distal end
24: second open tip
3: sucker
30: first end, top of sucker 3
31: first open tip of sucker 3
32: second end, base of sucker 3
33: second open tip of sucker 3
4: connection means
5: negative pressure generator
6: trocar
7: support arm
8: surgical table
9: surgical instrument
90: protruding end of surgical instrument 9
80: thoracic wall
80′: abdominal wall
200: anatomic organ

### Instrument Adapted for Cardiac Surgery

51: instrument
52: main rod
520: inner wall of main rod 52
521: outer wall of main rod 52
522: distal end of main rod 52
523: proximal end of main rod 52
700: robotic arm
300: robotic system
8: surgical table
400: 3D-imaging system
50: heating and cutting means
500: cutting elements, electrodes
500′,500″,500′″: electrodes
510: support means, dome structure
530: base of the dome structure 510
531: tip of the dome structure 510
512: outer surface of the dome structure 510
513: inner surface of the dome structure 510
540: connection zone
401: joint
S: spreading angle
402,403,404: actuators
503,501: flexible layers of dome structure 510
502: inner space
303: continuous metallic strip
305: sensors
520′,520″,520′″: holes
100: heart chamber
180: thoracic cavity

### Instrument Adapted for Hepatic Surgery

81: instrument
82: main rod
820: proximal end of main rod 82
821: distal end of main rod 82
B: main axis of main rod 82
83: secondary rod
830: proximal end of secondary rod 83
831: distal end of secondary rod 83
B′: main axis of secondary rod 83
84,84′: connection arms
85,85′: primary monopolar electrode
86: secondary monopolar electrode
87: bipolar electrode
813,814,815,816: actuators
87: bipolar electrode
870: first pole needle of bipolar electrode 87
871: second pole needle of bipolar electrode 87
B″: main axis of needle 870
B′″: main axis of needle 871
600: liver
700: robotic arm
8: rigid support, surgical table
4: connection means

1-38. (canceled)

39. Surgical instrument (51) adapted to cardiac surgery, and in particular to atrial defibrillation comprising insertion means for insertion inside the heart chamber (100), and cutting means (50) connected at a connection zone (540) to said insertion means, for creating lesions inside the heart chamber (100), said instrument (51) being such that both its translation and rotation movements are controlled by a robotic system (300) preferably coupled to a 3D-imaging system (400),
wherein the insertion means correspond to a rigid elongated stem (52) delimited by an outer wall (521), with a main axis (A), and having a proximal end (523) and a distal end (522), said proximal end (523) being connected to the robotic system (300), while the distal end (522) is free;
and wherein the cutting means comprise a flexible spreadable support structure (510) with an inner surface (511) and an outer surface (512), and an electrode mesh or network (500,500′,500″, . . . ) arranged on the outer surface (512) of said support structure (510).

40. The instrument according to claim 39, wherein the spreadable support structure (510) corresponds to a dome structure having a tip (531) and a base (532), said base (532) being free and said tip being connected at the connection zone (540) to the outer wall (521) of the stem (52).

41. The instrument according to claim 40, wherein the dome structure (510) is subdivided into dome sections able to selectively adopt a rest configuration for which all the dome sections are folded up along the outer wall (521) of the stem (522) and a plurality of working configurations for which at least one dome section selectively spreads from the stem (52) according to a spreading angle (S) defined by the main axis (A) of the stem (52), the connection zone (540) and the base (521) of the dome structure (510).

42. The instrument according to claim 40, wherein the electrode mesh or network comprises a plurality of parallel electrodes (500,500′,500″, . . . ) arranged both radially and circularly on the outer surface (512) of said dome structure (510), and activable selectively by the robotic system (300).

43. Method for performing an atrial defibrillation using the instrument (51) according to claim 47, comprising the following steps:
making a small incision in the thoracic wall (80) of the patient so as to introduce guiding means (1) inside the patient's cavity until the outer surface of the heart chamber, whereon said guiding means (1) are placed;
stabilising said guiding means (1) by attaching them to an immobile surface such as a surgical table (7);
under the control of the robotic system (300), passing the instrument (51) through said guiding means (1) by its distal end, with the dome structure (510) in rest configuration, until said instrument reaches the heart chamber and penetrates inside the heart chamber;
positioning the instrument (51) inside the heart chamber relatively to the atrial wall and following a predefined sequence of translation and rotation movements of the stem (52) and of the dome structure (510) corresponding to a sequence of working configurations for the dome structure (510);
coupling said sequence with a predefined activation sequence wherein different electrodes (500,500′,500″, . . . ) of the electrode network are selectively activated, so as to create selective lesions at precise locations in the atrial wall, said lesions being able to stop the electrical impulses associated to atrial fibrillation.

44. The method according to claim 43, wherein a surgical protocol is pre-established by taking a series of 3D images of the heart with the 3D-imaging system (400) and treating said images with the robotic system (300) so as to predefine the sequence of rotations and translations to give to the instrument (51) as well as the activation sequence of the electrodes (500,500′,500″) in the electrode network.

45. The method according to claim 43, wherein the 3D-imaging system (400) coupled to the robotic system (300) takes a series of 3D-images as a function of time, preferably in real time, thereby allowing a monitoring of the surgical procedure.

46. Surgical instrument (81) adapted for hepatic surgery and comprising insertion means for insertion inside a target organ, and heating means for coagulating specific tissue regions inside said target organ, said heating means being connected to said insertion means, said instrument (81) being capable of translation and rotation movements controlled by a robotic system preferably coupled to a 3D-imaging system and being adapted for intra-hepatic surgery.

47. The instrument according to claim 46, wherein the insertion means correspond to a rigid elongated rod (82) with a main axis (B), a centre of gravity (O), a proximal end (820) and a distal end (821), said proximal end (820) being connected to the robotic system, while the distal end (821) is free.

48. The instrument according to claim 46, wherein the heating means comprise at least (i) a secondary rigid rod (83) articulated on the main rod (82) via connection means (84) and provided with a main axis (B′), a proximal end (830) and a distal end (831), and (ii) at least one electrode (85,85′,86,87) activable by the controlling means, preferably by radiofrequency.

49. The instrument according to claim 46, wherein the heating means comprise at least one bipolar electrode (87), articulated on the main rod (82) via connection means (84,84′), preferably consisting of one first needle (870) and a second needle (871), each of said needle (870,871) being defined by a main axis (B″,B′″), and activable by the controlling means, preferably by radiofrequency.

50. The instrument according to claim 50, comprising two primary monopolar electrodes (85,85′) which are at least part of the secondary rod (83) and are activable selectively by the controlling means, preferably by radiofrequency.

51. The instrument according to claim 48, further comprising at least one secondary monopolar electrode arranged at the distal end (821) of the main rod (82) and activable selectively from said primary electrodes (85,85′) or said bipolar electrode (87).

52. The instrument according to claim 46, wherein the main rod (82) possesses six degrees of freedom, four of them being able to be blocked in operating conditions by the controlling means so as to allow only two degrees of freedom, one translation along and one rotation around its main axis (B).

53. The instrument according to claim 46, wherein the secondary rod (83) has its main axis (B′) parallel to the main axis of the main rod (82) and presents two degrees of freedom, one translation along and one rotation around its main axis (B′) so that the height and the distance of the secondary rod (83) relatively to the main rod (82) can be adjusted by the controlling means, the distance being adjustable at a value varying from O to a maximum value.

54. The instrument according to claim 46, wherein the bipolar electrode (87) presents different degrees of freedom, one rotation around their main axis (B″,B′″) for each of said first and second needles (870,871) and one translation along said axis (B″,B′″) so that the distance of the needles (870,871) relatively to the main rod (82) can be adjusted by the controlling means, said distance being adjustable at a value varying from O to a maximum value.

55. The instrument according to claim 46, said instrument (81) being able to adopt one rest configuration and at least one working configuration, each of said configurations being defined by a different relative position of the insertion means and/or the heating means, the instrument being not functional in rest configuration but being functional in working configuration.

56. The instrument according to claim 55, wherein when in rest configuration, the secondary rod (83) or the bipolar electrode (87) is folded up inside the main rod (82) (distance main rod (82)/secondary rod (83) or main rod (82)/bipolar electrode (87) equal to 0), and all the electrodes (85,85′,86) are unactivated.

57. The instrument according to claim 55, wherein in a working configuration, the secondary rod (83) spreads out from the main rod (82), its main axis (B′) being parallel to the one (B) of the main rod (82) and distanced to it of a certain distance greater than 0 and at least of the electrode (85,85′,86) is activated.

58. The instrument according to claim 55, wherein in a working configuration, the bipolar electrode (87) spreads out from the main rod (82), the main axis (B″,B′″) for each of said first and second needles (870,871) being parallel to the main axis (B) of the main rod (82) and distanced to it of a certain distance greater than 0 and at least of the electrode (87,86) is activated.

59. A method for coagulating an intra-hepatic tumour of a certain shape, using the instrument according to claims 46, comprising the following steps:
making a small incision in the abdominal wall of the patient so as to introduce guiding means inside the patient's cavity until the outer surface of the liver, whereon said guiding means are placed;
stabilising said guiding means by attaching them to an immobile surface such as a surgical table;
under the control of the robotic system, passing the instrument through said guiding means by its distal end, with the instrument in rest configuration, until said instrument reaches the liver and penetrates inside the hepatic parenchyma;
positioning the instrument inside the hepatic parenchyma relatively to the hepatic wall and following a predefined sequence of translation and rotation movements of the main rod and the secondary rod corresponding to a sequence of working configurations;
coupling said sequence with a predefined activation sequence wherein different electrodes of the electrode network (first and second electrodes) are selectively activated, so as to lead to a tissue coagulation at precise target locations in the liver corresponding to tumour tissues.

60. The method according to claim 59, wherein a surgical protocol is pre-established by taking a series of 3D images of the liver and of the tumour with the 3D-imaging system and treating said images with the robotic system so as to predefine the sequence of rotations and translations to give to the instrument as well as the activation sequence of the electrodes in the electrode network.

61. The method according to claim 59, wherein the 3D-imaging system coupled to the robotic system takes a series of 3D-images as a function of time, preferably in real time, thereby allowing a monitoring of the surgical procedure.
